SEQUENCE LISTING (8 sequences)

SEQ ID NO: 1 - DNA, 18 bp, Pseudomonas putida
agcatcctcatccacaac

SEQ ID NO: 2 - DNA, 18 bp, Pseudomonas putida
gccgtgtctatctgaagg

SEQ ID NO: 3 - DNA, 19 bp, Escherichia coli
gtggcgcattgattcttgg

SEQ ID NO: 4 - DNA, 20 bp, Escherichia coli
ccggctgattttttcgatga

SEQ ID NO: 5 - DNA, 20 bp, Enterobacter cloacae
tgttgccgcgcagcatgtag

SEQ ID NO: 6 - DNA, 20 bp, Enterobacter cloacae
tgaccggcgacaaccagaag

SEQ ID NO: 7 - DNA, 22 bp, Enterobacter aerogenes
gccatgccagaaggttatgttc

SEQ ID NO: 8 - DNA, 18 bp, Enterobacter aerogenes
agcatcctcatccacaac

(19) United States
(12) Patent Application Publication
(10) Pub. No.: US 2019/0234947 A1
(43) Pub. Date: Aug. 1, 2019

(54) SENSOR DEVICE FOR DETECTING TARGET MICROORGANISM IN TAP WATER IN REAL TIME

(71) Applicant: Industry-Academic Cooperation Foundation, Yonsei University, Seoul (KR)

(72) Inventors: Joonhong PARK, Seoul (KR); Seongchan JUN, Seoul (KR); Jihyun KIM, Seongnam-si (KR); Youngmo JUNG, Seoul (KR)

(73) Assignee: Industry-Academic Cooperation Foundation, Yonsei University, Seoul (KR)

(21) Appl. No.: 16/197,609

(22) Filed: Nov. 21, 2018

(30) Foreign Application Priority Data
Jan. 26, 2018 (KR) 10-2018-0009910
Nov. 13, 2018 (KR) 10-2018-0139263

(51) Int. Cl. G01N 33/569; G01N 27/06; G01N 27/22

(52) U.S. Cl. CPC G01N 33/56916; G01N 27/22; G01N 27/06

(57) ABSTRACT

A sensor device for detecting a target microorganism in tap water in real time, the sensor device includes: a substrate; and a microorganism detection unit configured to detect the target microorganism. A microorganism mounting unit and a signal recognition unit are provided on the microorganism detection unit, and a hydrothermal synthesis unit and a signal disturbance blocking unit are provided on the signal recognition unit to detect an electrochemical reaction occurring in the microorganism mounting unit as an impedance signal value and to measure or detect the target microorganism.

### ACKNOWLEDGEMENTS

This research was supported by the following Project sponsored by the Ministry of Environment.

Project No.: 201600213005

Government Department: Ministry of Environment

Project Name: Global Top Project

Research Title: Development of equipments for improving the safety of underground waterworks facilities

Research Management Institution: Seoyong Engineering Co., Ltd.

Research Period: From Aug. 10, 2016 to Apr. 30, 2021

### CROSS-REFERENCE TO RELATED APPLICATIONS

This application claims priority to and the benefit of Korean Patent Application Nos. 10-2018-0009910 filed on Jan. 26, 2018 and 10-2018-0139263 filed on Nov. 13, 2018, the disclosure of which are incorporated herein by reference in their entirety.

### BACKGROUND

The present invention relates to a sensor device for detecting a target microorganism in tap water in real time, and more specifically, to a device capable of measuring or detecting a target microorganism in tap water or cleaning water in real time based on an electrochemistry impedance spectroscopy.

According to a drinking water quality standard (2015) of the Korean Ministry of Environment, in tap water, a drinking water public facility, and drinking groundwater, general bacteria should be present at a concentration of less than 100 CFU/ml when measured through a pour plate method, and total coliforms, fecal coliforms, and Escherichia colis should not be detected when measured through a multiple tube fermentation method, a membrane filtration method, or an enzyme substrate method.

Since such official test methods are culture-based methods, it is important to find that active bacteria are present in tap water even when the active bacteria are present at a concentration less than or equal to a standard value or are not detected.

Since the culture-based method has low detection consistency according to environmental characteristics of a sample, verification of a novel method is generally performed through a molecular biological method, i.e., a quantitative polymerase chain reaction (PCR).

In addition, since it is possible to diagnose completeness of cleaning of an indoor water supply pipe and a risk and the like due to residual microorganisms through a detection and analysis of bacteria in washing water, in order to develop a biosensor suitable for such applications, there is a need to set a detection limit.

### PRIOR ART DOCUMENT

### Patent Document

(Patent Document 1) Korean Patent Publication No. 2004-0012854

### SUMMARY

In a sensor device for detecting a target microorganism in tap water in real time according to the present invention, the present inventors have endeavored to overcome the above problems and thus discovered a device capable of measuring or detecting an active bacterium or microorganism even when the active bacterium or microorganism is present at a concentration less than or equal to a standard value or is not detected in the tap water, thereby finally completing the present invention.

Therefore, the present invention is directed to providing a device capable of measuring or detecting a target microorganism in tap water or cleaning water in real time.

On the other hand, the present invention provides a sensor device for measuring or detecting a target microorganism in tap water or cleaning water in real time, the sensor device including a detection unit configured to undergo an antigen-antibody reaction with the target microorganism and a signal extraction unit configured to monitor an electrochemical reaction occurring through the antigen-antibody reaction by converting the electrochemical reaction into an impedance value, wherein the sensor has an array form in which single detection units are arranged in parallel and thus has a structure in which different antibodies are attached to the detection units, thereby detecting the target microorganism.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a cross-sectional view illustrating a sensor device for measuring or detecting a target microorganism in tap water or cleaning water according to the present invention;

FIG. 2 is a view illustrating that the target microorganism is detected or destroyed in the sensor device according to the present invention;

FIG. 3 is a block diagram illustrating a configuration of a system including the sensor device according to the present invention; and

FIG. 4 is a characteristic curve graph showing a response (impedance change) according to the number of bacteria of a biosensor according to the present invention.

### DETAILED DESCRIPTION

Hereinafter, the present invention will be described in more detail.

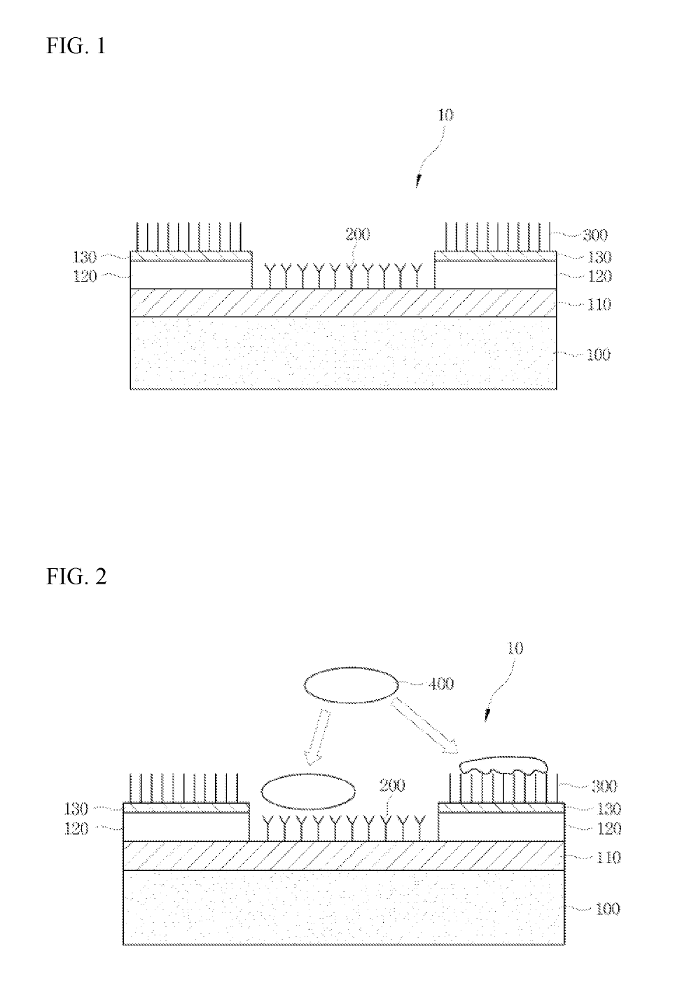

An embodiment of the present invention relates to a sensor device 10 for detecting a target microorganism in tap water in real time, the sensor device 10 including a substrate 100 and a microorganism detection unit 110 configured to detect a target microorganism, wherein a microorganism mounting unit 200 and a signal recognition unit 120 are provided on the microorganism detection unit 110, and a hydrothermal synthesis unit 130 and a signal disturbance blocking unit 300 are provided on the signal recognition unit 120 to detect an electrochemical reaction occurring in the microorganism mounting unit 200 as an impedance signal value and to measure or detect the target microorganism (see FIG. 1).

In an embodiment of the present invention, the microorganism includes a bacterium, Escherichia coli (E. coli), and the like, but the present invention is not limited thereto.

In an embodiment of the present invention, the microorganism detection unit 110 provided on the substrate 100 is composed of a silicon dioxide wafer (Sift wafer). The microorganism mounting unit 200 is formed at a center of the microorganism detection unit 110. The signal recognition unit 120 is formed along a circumference of the microorganism mounting unit.

The substrate 100 may be made of silicon (Si), but the present invention is not limited thereto.

The microorganism detection unit 110 is an antibody-based biosensor and includes the microorganism mounting unit 200 including an antibody on an upper portion thereof which is bonded to a microorganism to cause a reaction. A target microorganism 400 is attached to the antibody of the microorganism mounting unit 200 to cause a selective reaction, and the target microorganism may be detected through the reaction (see FIG. 2).

When the target microorganism 400 is selectively bonded to the antibody, a change in dielectric characteristic or a change in characteristic of an electric double layer occurring at a binding site is electrically measured.

The target microorganism 400 has a biological tissue structure and thus has electrical impedance which is changed according to a frequency. A tissue has both resistive and capacitive characteristics which cause complex electrical impedance, and magnitude and frequency dependence of the impedance are changed according to a tissue structure. When impedance of the target microorganism is measured within a frequency range, a characteristic of a biological tissue, i.e., a spectrum, is generated. Accordingly, a change in impedance spectrum is directly related to a change in elemental characteristic of a tissue.

In a conventional impedance measurement sensor, signal disturbance occurs in a signal recognition unit due to contact with a target microorganism, resulting in a difficulty in high resolution measurement. However, in the present invention, the signal disturbance blocking unit 300 destroys a structure of the target microorganism to minimize signal disturbance. As a result, the target microorganism is selectively bonded to the antibody only in the microorganism detection unit.

In an embodiment of the present invention, the signal recognition unit 120 includes a metal sensor electrode in the form of an interdigitated array in which single detection units are arranged in parallel.

Gold (Au) or the like may be used as the metal, but the present invention is not limited thereto.

In an embodiment of the present invention, the hydrothermal synthesis unit 130 is made of graphene, and the graphene is hydrothermally synthesized to form a plurality of metal nanowires. The nanowires constitute the signal disturbance blocking unit 300.

Such a bonding structure of graphene and metal nanowires may contribute to improvements in impedance signal stability and reactivity.

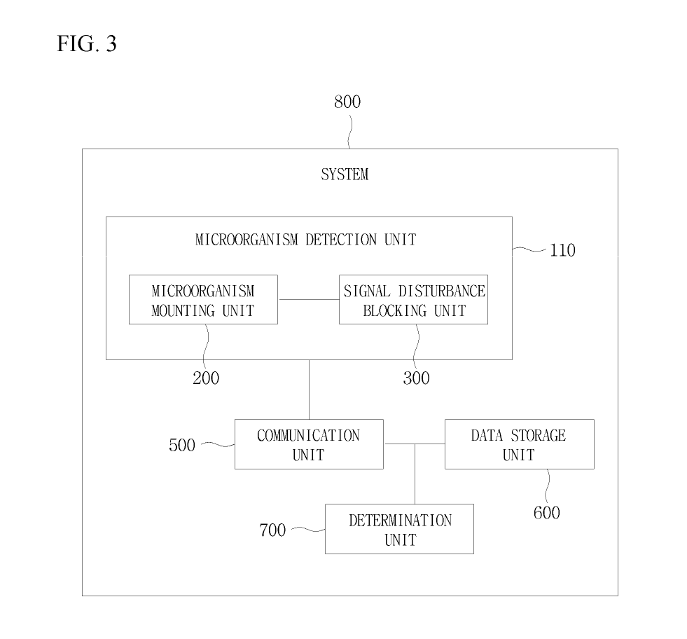

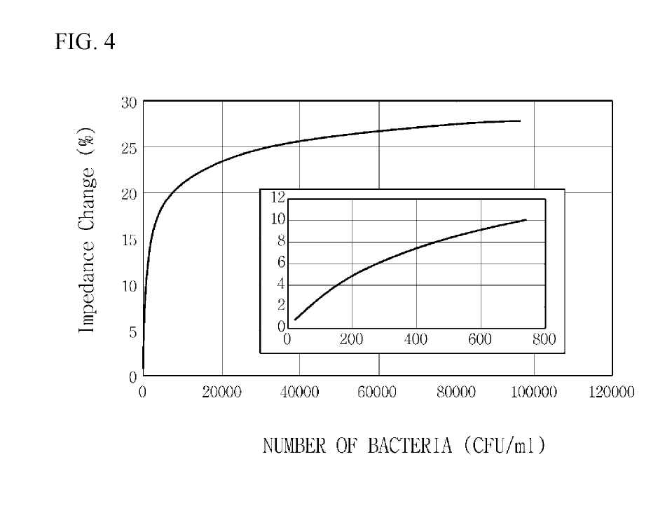

An embodiment of the present invention relates to a system 800 for measuring or detecting a target microorganism in tap water or cleaning water, the system 800 including a sensor device 10, a communication unit 500, a data storage unit 600, and a determination unit 700 (see FIG. 3).

Hereinafter, the present invention will be described in more detail through examples. It will be obvious to a person having ordinary skill in the art that these examples are illustrative purposes only and are not to be construed to limit the scope of the present invention.

The number of bacterial 16S rRNA copies conforming to a drinking water quality standard of Koreas Ministry of Environment was derived based on experimental results of the present inventors, and a detection limit target of a biosensor was set such that the biosensor performed detection to a level of 10% of the number of the bacterial 16S rRNA copies (see Table 1).

TABLE 1

| | The number of 16S | Detection limit target |
|---|---|---|
| Target | rRNA genes following | of novel sensor: |
| microorganism | drinking water quality | the number of 16S |
| item | standard¹⁾ | rRNA genes²⁾ |
| General bacterium | 1,000 16S copies/ml | >100 16S copies/ml |
| (total bacterium) | | |
| E. coli | 10 16S copies/100 ml | >1 16S copies/100 ml |

An object of the present invention, i.e., a second sample was washing water obtained in a process of cleaning an indoor water supply pipe. It was possible to diagnose completeness of the cleaning of the indoor water supply pipe and a risk and the like due to residual microorganisms through a detection and analysis of bacteria in the washing water. Therefore, in order to develop a biosensor suitable for such applications, it was necessary to set the detection limit target.

To this end, a range of a measured value in the washing water was analyzed based on data obtained by analyzing about 30 samples of the washing water in the indoor water supply pipe. The range of the measured values was shown in Table 2 below. A significantly large number of bacteria was detected as compared with tap water, and the range of the measured values was considerably wider. When a new sensor was capable of detecting 10% of a minimum value among the measured values in the washing water, it was determined that the new sensor was sufficient. When the new biosensor was used to analysis the washing water, detection limit targets were set in Table below.

TABLE 2

| Target | | Detection limit target |
|---|---|---|
| microorganism | Measured value in | of novel sensor: the |
| item | washing water | number of 16S rRNA genes¹⁾ |
| General bacterium | 10⁴-10⁶ CFU/ml ²⁾ | >1,000 CFU/ml |
| (total bacterium) | 10⁷-10¹¹ 16S copies/ml ³⁾ | >10⁶ 16S copies/ml |
| E. coli | 10²-10⁴ CFU/ml ⁴⁾ | >10 CFU/ml |
| | 10³-10⁵ 16S copies/ml ⁵⁾ | >100 16S copies/ml, |

wherein 1) a target is set to a level of 10% of a minimum value of the measured values in the washing water,

2) measurement is performed through a total colony count-pour plate method of a drinking water quality standard,

3) the number of live total bacterial 16S rRNA genes is measured after a propidium monoazide (PMA) pretreatment,

4) measurement is performed through an E. coli-membrane filtration method of a drinking water quality standard, and

5) the number of live E. coli 16S rRNA genes is measured after a PMA pretreatment.

In the present invention, detection limit evaluation was performed on a general bacterium detection biosensor (1) in which a specific target microorganism antibody is not installed and an E. coli detection biosensor (E. coli specific sensor) (2) in which a specific target microorganism (E. coli) antibody is installed.

Whether a microorganism in a sample was detected through an impedance reaction of a sensor was performed by the following procedure. Rather than directly applying a sample of tap water, E. coli K12 purely cultured in a known culture medium was prepared to have different concentrations and introduced into a microorganism detection sensor, and impedance generated in the sensor was measured. In this case, the lowest microorganism concentration, in which a change in impedance is statistically significant at 95% level (p-value<0.05) as compared with an impedance profile of only phosphate buffered saline (PBS) as a blank sample, was calculated as a detection limit.

As a result, a detection limit of the general bacterium detection biosensor was found to be about 10 CFU/ml of a pour plate method. Considering that a detection limit of conventional microorganism sensor technology is in a range of 50-1,000 CFU/ml (Sungkyunkwan University, 2009 and Korean Rural Development Administration, 2014), the sensor developed in the present invention is determined to have excellent sensitivity.

Since the measured detection limit shows a statistically significant difference from a drinking water quality standard of 100 CFU/ml (p-value<0.001), the developed sensor may be applicable to the drinking water quality standard. A verification result using a quantitative polymerase chain reaction (PCR) also showed that a detection limit was significantly different from 1,000 copies/ml, which is the number of total bacteria 16S rRNA genes (Table 3) which corresponds to the number of general bacteria of the drinking water quality standard. Therefore, the developed sensor has proved to have a detection limit characteristic suitable for detecting general bacteria of the drinking water quality standard.

TABLE 3

| Bacteria | Measured | Detection limit | Detection limit target |
|---|---|---|---|
| quantification | detection | target (drinking | (minimum measured |
| method | limit | water standard) | value of washing water) |
| Membrane filtration | 10 ± 10 | — | 1,000 |
| method | | (not detected) | (10,000) |
| (CFU/ml) | | | |
| 16S Q-PCR | 100-1,000 | 10¹⁾ | 10⁶ |
| (16S copies/ml) | | (100) | (10⁷), |

wherein 1) a value is converted into the number of general bacteria (total bacteria) 16S rRNA genes according to a drinking water quality standard.

Since the results of Table 3 above show that a PBS buffer solution is used as a background solution, it is presumed that there will be a difference in a sensor response between tap water and the PBS buffer solution in terms of a background effect. In the sensor used in the present invention, as in PBS, as an ion concentration becomes higher, an interference effect to an impedance signal becomes higher. However, when an ionic concentration is very low as in tap water or drinking water, the interference effect is very low. In this respect, a detection limit in tap water is expected to be lower than that in Table 3. Additional experiments on the detection limit in the tap water are under way (to be confirmed in the final announcement). Current results are obtained by introducing small amounts (0.01-0.05 ml) into the sensor. When a pretreatment of concentrating a sample is performed, it is possible to detect general bacteria of the same sample even at a lower concentration.

As for evaluation of usability for detection of general bacteria in washing water, since a detection limit target of the washing water is higher than a detection limit target of a drinking water, it was determined that a general bacteria detection sensor achieving the detection limit target of the drinking water target sufficiently achieved the detection limit target of the washing water. However, unlike tap water, the washing water contains a large amount of materials formed due to corrosion and scale.

In the present invention, a sensor using impedance measurement, which has relatively high reactivity even at a low microorganism concentration (CFU) so as to detect a selected target microorganism and secures economical feasibility as compared with a conventional analysis method has been designed and implemented. The sensor includes a detection unit configured to undergo an antigen-antibody reaction with a target microorganism and a signal extraction unit configured to monitor an electrochemical reaction occurring through the antigen-antibody reaction by converting the electrochemical reaction into an impedance value. The sensor has an array form in which single detection units are arranged in parallel and thus has a structure in which different antibodies are attached to the detection units, thereby detecting a target microorganism.

The sensor is implemented through a multi-stage photo-lithography process. Detection electrode patterns (at intervals of 3 μm to 10 μm, comb electrode structure) were implemented based on the photo-lithography process on a Sift substrate which is generally used in a semiconductor process, and a process was additionally performed by attaching a polydimethylsiloxane (PDMS) structure to passivate an electrode portion excluding an antibody-attached surface. This may prevent a signal disturbance due to reaction elements except for a target microorganism. Detection electrodes were implemented using gold (Au), and a productized antibody was attached to a surface of Sift between the detection electrodes by using a reaction agent. The comb electrode structure of the detection unit of the sensor was designed by adjusting a distance between both electrodes in order to improve sensitivity of the sensor to the target microorganism. Since the sensitivity of the sensor was determined based on a shape of an electrode and a degree of an electrical response to a target material, the comb electrode structure was designed considering a concentration range, a size, and an electrical characteristic of various target microorganisms. In addition, in order to increase sensitivity to a sample with a low microorganism concentration, the detector was designed to have a large area, thereby increasing a possibility by which the target microorganism selectively reacts with an antibody on a surface of the detection unit even at a low concentration.

### Experimental Example 1

Analyzing all members of a microorganism community in tap water and washing water is the best method, but the analyzing is both costly and time consuming. Thus, it has been determined to add individual variability in the microorganism community as an item for measuring ecological factors. A simple method of measuring the ecological factors was as follows: 16S rRNA genes were amplified through a PCR and were analyzed through a terminal restriction fragment length polymorphism (T-RFLP) based on simple enzyme digestion, and alpha diversity at a class level was measured by the Shannon index. As other biological items, four species of indicator bacteria representing E. colis, fecal coliforms, total coliforms, and general bacteria were selected, and quantitative numbers thereof were measured through a quantitative PCR (qPCR) (see Table 4).

Quantitative detection of microorganisms using the qPCR was performed as follows:

1) an appropriate qPCR primer and PCR cycle of a target microorganism were selected,

2) primers were prepared,

3) deoxyribonucleic acid (DNA) products were formed by performing PCR amplification on the primers prepared using a corresponding microorganism,

4) PCR amplification was concurrently performed using a sample of which the number of copies is known, and

5) the number of gene copies of the target microorganism was calculated.

TABLE 4

| Microorganism | Primers (5′-3′) | Gene | Reference |
|---|---|---|---|
| Pseudomonas | xylE-F: AGCATCCTCATCCACAAC (SEQ ID NO: 1) | xylE | Chang et al., |
| putida | xylE-R: GCCGTGTCTATCTGAAGG (SEQ ID NO: 2) | | 2009 |
| Escherichia | tir-F: GTGGCGCATTGATTCTTGG (SEQ ID NO: 3) | tir | Higgins et al., |
| coli | tir-R: CCGGCTGATTTTTTCGATGA (SEQ ID NO: 4) | | 2003 |
| Enterobacter | HycE-F: TGTTGCCGCGCAGCATGTAG (SEQ ID NO: 5) | HycE | Notbert Acs et |
| cloacae | HycE-R: TGACCGGCGACAACCAGAAG (SEQ ID NO: 6) | | al., 2005 |
| Enterobacter | GFP-F: GCCATGCCAGAAGGTTATGTTC (SEQ ID NO: 7) | GFP-F | K.Verthe et al., |
| aerogenes | GFP-R: CAAACTTGACTTCAGCTCTGGTCTT (SEQ ID NO: 8) | | 2004 |

### Experimental Example 2: Characteristic Curve Analysis of Sensor Basal Response

Characteristics of an impedance change response to a bacterial concentration (or the number) of a basic sensor developed in the present invention were examined.

As a result, two suggestions are propsed. First, as the number of bacteria is increased, a response of the sensor is increased proportionally, and a change rate of impedance tends to be saturated near 30%. That is, the response of the sensor tends to be saturated when the number of bacteria is greater than a range of 40,000 CFU/ml to 60,000 CFU/ml. Second, there is a linear correlation between the number of bacteria and a change rate of the impedance of the sensor in a range of 800 CFU/ml or less in which the number of the bacteria is relatively small (see FIG. 4).

Since an object of the present invention, i.e., the number of bacteria of drinking water, is small, the linear correlation appearing at a level of the small number of the bacteria suggests a possibility of quantitative detection capability of the sensor developed in the present invention. On this basis, the quantitative detection capability of sensors manufactured in the present invention was experimentally evaluated by focusing on the number of bacteria less than or equal to 1,000 CFU/ml.

### Experimental Example 3: Quantitative Detection Capacity of General Bacterium Detection Biosensor

It was experimentally evaluated whether a general bacterium detection biosensor developed in the present invention was capable of quantitatively measuring the total number of bacteria in a sample. Measured values through a conventional pour plate method (Luria Bertani (LB) broth) and a non-specificity biosensor developed in the present invention with respect to samples having various total numbers of bacteria were compared with each other and were analyzed by using a culture result of a single strain including only E. coli K12 in a PBS solution.

As a result, it was confirmed that a change in electrical signal of a sensor, i.e., a change in impedance was quantitatively correlated with the total number of bacteria (R²=0.9584). This indicates that a quantitative analysis is possible because a response of the biosensor is increased as the number of general bacteria is increased in a sample.

### Experimental Example 4: Evaluation of Specificity Detection Capacity of Target Microorganism in Microorganism Community

In order to evaluate whether an E. coli antibody-attached biosensor selectively detected only E. coli and did not detect non-E. coli, mock communities were prepared as shown in Table 5 by selecting E. coli K12 as an indicator bacterium of E. coli, selecting a non-E. coli bacterium, i.e., Pseudomonas putida as an indicator bacterium, and mixing single strains thereof at different ratios.

TABLE 5

| | Sample | Sample | Sample | Sample | Sample |
|---|---|---|---|---|---|
| Specification | #1 | #2 | #3 | #4 | #5 |
| Concentration of E. coli | 4,800 | 4,800 | 4,800 | 4,800 | 4,800 |
| K12 (CFU/ml) | | | | | |
| Pseudomonas putida | 6,900 | 3,450 | 0 | 690 | 345 |
| (CFU/ml) | | | | | |

Even when a concentration of the E. coli K12 was fixed to 4,800 CFU/ml and a concentration of the Pseudomonas putida was variously changed, consistent measurement results were shown. The results prove that the developed sensor has the capability to consistently detect a target bacterium, E. coli, regardless of an influence from bacteria other than the target bacterium.

Even when an active bacterium or microorganism is present at a concentration less than or equal to a standard value or is not detected in the tap water, the active bacterium or microorganism can be monitored or detected in real time by using a device for detecting a microorganism according to the present invention.

Although particular embodiments of the present invention have been shown and described, it will be understood by those skilled in the art that it is not intended to limit the present invention to the preferred embodiments. It will be obvious to those skilled in the art that various changes and modifications may be made without departing from the spirit and scope of the invention.

The scope of the present invention, therefore, is to be defined by the appended claims and equivalents thereof.

1. A sensor device for detecting a target microorganism in tap water in real time, the sensor device comprising:
a substrate; and
a microorganism detection unit configured to detect the target microorganism,
wherein a microorganism mounting unit and a signal recognition unit are provided on the microorganism detection unit, and a hydrothermal synthesis unit and a signal disturbance blocking unit are provided on the signal recognition unit to detect an electrochemical reaction occurring in the microorganism mounting unit as an impedance signal value and to measure or detect the target microorganism.

2. The sensor device of claim 1, wherein the microorganism includes at least one selected from the group consisting of Pseudomonas putida, Escherichia coli, Enterobacter cloacae, and Enterobacter aerogenes.

3. The sensor device of claim 1, wherein the microorganism detection unit is composed of a silicon dioxide wafer, the microorganism mounting unit is formed at a center of the microorganism detection unit, and the signal recognition unit is formed along a circumference of the microorganism mounting unit.

4. The sensor device of claim 1, wherein the microorganism mounting unit includes an antibody which is bonded to a microorganism to cause a reaction.

5. The sensor device of claim 4, wherein, when the antibody is bonded to the microorganism, the target microorganism is detected through a change in dielectric characteristic or a change in characteristic of an electric double layer occurring at a binding site.

6. The sensor device of claim 1, wherein the signal recognition unit includes a metal sensor in a form of an interdigitated array in which single detection units are arranged in parallel.

7. The sensor device of claim 1, wherein the thermal synthesis unit is made of graphene, the graphene is hydrothermally synthesized to form a plurality of metal nanowires, and the nanowires constitute the signal disturbance blocking unit.

8. The sensor device of claim 1, wherein the signal disturbance blocking unit destroys a microorganism not in contact with the microorganism mounting unit and blocks a signal recognized in the microorganism detection unit from being disturbed.

9. A system for detecting a target microorganism in tap water in real time, the system comprising the sensor device, a communication unit, a data storage unit, and a determination unit according to claim 1.
